(19) United States
(12) Patent Application Publication
(10) Pub. No.: US 2024/0060044 A1
(43) Pub. Date: Feb. 22, 2024

(54) A NOVEL METHOD OF GENERATING T CELLS FROM PERIPHERAL BLOOD PRECURSORS AND THEIR USES THEREOF

(71) Applicant: Senthilkumar NATESAN, Ahmedabad (IN)

(72) Inventor: Senthilkumar NATESAN, Ahmedabad (IN)

(21) Appl. No.: 18/547,163

(22) PCT Filed: Jan. 5, 2022
(86) PCT No.: PCT/IB2022/050055

§ 371 Date: Aug. 21, 2023

(30) Foreign Application Priority Data
Feb. 25, 2021 (IN) 202121007955

(51) Int. Cl. C12N 5/0783; A61K 39/00

(52) U.S. Cl. CPC C12N 5/0636; A61K 39/4611; A61K 2039/5158; C12N 2501/2302; C12N 2510/00

(57) ABSTRACT

The present invention describes a novel method of generating the T cells from the cell fraction obtained from human peripheral blood mononuclear cells or peripheral blood progenitor cells or peripheral blood precursors. The present invention also provides a novel method of generating CAR T cells (chimeric antigen receptor) using the T cells generated from specific cell fraction and a novel method of generating engineered or modified T cell ex vivo.

### FIELD OF THE INVENTION

The present invention provides a novel method to generate T cells from the peripheral blood progenitor cells, in vitro, which can be used for developing novel therapies, diagnostic assays, disease model and drug screening.

### BACKGROUND OF THE INVENTION

Developmental biology of T cells in human is still not fully understood and the current knowledge is based on the mice model and thymus organ culture. There is lack of understanding on T cell progenitor or precursor cells emigrating the bone marrow and present in the circulation prior to enter into the thymus or elsewhere in the extra-thymic sites. T cell generation in human is still poorly understood area and all the current understanding of T cell development is based on the studies done in mice and its relevance to human is questionable. Most T cell developmental studies were carried out using the thymus and extra-thymic T cell development is also poorly understood, even though there are strong evidences for extra-thymic T cell development in human following surgical removal of thymus. Understanding the T cell progenitor that emigrate bone marrow and travel in the blood circulation prior to reach thymus when thymus is present or elsewhere when thymus is absent. For studying the T cell development in human, there is a need of simple and reliable in vitro model which could help to understand the HIV and other infectious diseases as well as for developing novel gene therapy approaches through genome editing methods for cancers and genetic disorders. The T cells can be obtained from methods available in art and it can be derived or obtained from many source known in the art. For example, T cells can be differentiated in vitro from a stem cell population using fetal thymic organ culture (FTOC) model system. This approach relies on the seeding of human hematopoietic stem cells (HSCs) and/or their progeny into host thymic lobes or thymic fragments, typically of mouse origin. An in vitro approach that makes use of the OP9 bone marrow stromal cell line expressing the Notch receptor ligand Delta-like-1 (0P9-DL1) have also been shown to support the generation of large numbers of human progenitor T cells from HSCs. These methods of T cell generation may not reflect the natural mechanisms of T cell generation in the human body. The donor can be a subject, e.g., a subject in need of an anti-cancer treatment. T cells can be obtained from, e.g., peripheral blood mononuclear cells, bone marrow, lymph node tissue, cord blood, thymus tissue, and tissue from a site of infection, ascites, pleural effusion, spleen tissue and tumours. In addition, the T cells can be derived from one or more T cell lines available in the art. T cells can also be obtained from a unit of blood collected from a subject using any number of techniques known to the skilled artisan, such as FICOLL™ separation and/or apheresis. T cells can also be obtained from an artificial thymic organoid (ATO) cell culture system, which replicates the human thymic environment to support efficient ex vivo differentiation of T-cells from primary and reprogrammed pluripotent stem cells.

Chimeric Antigen Receptor (CAR) T cell or CAR T cell is a novel therapeutic product targeting specific antigen on the cancer cells and killing them. It brings complete remission in majority of the patients who had relapsed and recurrent disease. Unlike the natural T-cell receptor, the chimeric antigen receptor in CAR T cells can recognize the antigen present on the cancer cells directly without any need for antigen presenting cells. In CAR T cell therapy, patients own T cells are genetically reprogrammed ex vivo and then they are injected back into the patient. The artificially expressed CAR on the T cells mediate targeted killing of cancer cells. CAR T cells are the first approved therapy where a modified living cell is recognised as a drug. It is approved for the treatment of B-cell Acute Lymphoblastic Leukaemia (B-ALL). It is shown to bring cure among patients suffering from relapsed B-ALL. It is targeted to eliminate the CD19 harboring leukemic cells of B-ALL patients who failed on all available lines of therapies and suffer from recurrent or relapsed disease.

EP3214091 provides an isolated nucleic acid sequence encoding a chimeric antigen receptor (CAR), wherein the CAR comprises an antigen binding domain, a transmembrane domain, a costimulatory signaling region, and a CD3 zeta signaling domain, wherein the CD3 zeta signaling domain comprises the amino acid sequence of SEQ ID NO: 24.

WO2014153270 provides compositions and methods for treating diseases associated with expression of CD19. The disclosure also relates to chimeric antigen receptor (CAR) specific to CD19, vectors encoding the same, and recombinant T cells comprising the CD19 CAR. The disclosure also includes methods of administering a genetically modified T cell expressing a CAR that comprises a CD19 binding domain.

WO2019232444A1 provides genome-edited chimeric antigen receptor T cells (CAR-T), which can be derived from a cytotoxic T cells, a viral-specific cytotoxic T cell, memory T cells, or gamma delta (y6) T cells and comprise one or more chimeric antigen receptors (CARs) targeting one or more antigens, wherein the CAR-T cell is deficient in one or more antigens to which the one or more CARs specifically binds. In particular, it relates to engineered mono, dual and tandem chimeric antigen receptor (CAR)-bearing T cells (CAR-T) and methods of immunotherapy for the treatment of cancer.

Among the antigens, CD19 has been the majorly researched antigen and has proved to be effective in treatment of Acute Lymphoblastic Leukemia (ALL) and Chronic Lymphocytic Leukemia (CLL). However, in the recent years, the research work is focused on other antigens as well.

Currently, there are two US-FDA approved CAR T cell therapeutic products Kymria for the treatment of relapsed/refractory Acute Lymphocytic Leukemia (ALL) and relapsed/refractory Diffuse Large B-Cell Lymphoma (DLBCL) indications and Yescarta for the treatment of certain relapsed/refractory large B-Cell non-Hodgkin lymphoma (NHL) available at a huge cost in the US.

Till date there is no any disclosure of any such method or process available to generate T cells from the specific fraction of cultured peripheral blood mononuclear cells in a simple and cost-effective way. The present invention describes the method to de novo generate T cells from the peripheral blood T cell precursors. The T cells generated using this approach can also be used for generation of CAR T cells which are useful for therapeutic purposes. Inventors of present invention have invented a novel method to de novo generate T cells and a novel method to generate of CAR T cells using them as described herein.

Objective of the Invention

The main objective of the present invention is to provide a novel method of generating T cells from the peripheral blood progenitor cells, in vitro.

Another objective of the present invention is to provide a novel method of generating chimeric antigen receptor, CAR T cells using the T cells from specific fraction of peripheral blood progenitor cells, in vitro.

### SUMMARY OF THE INVENTION

The main aspect of the present invention is to provide a novel method of generating T cells from the peripheral blood progenitor cells comprising the steps of
a) Collecting blood from donor in blood collection tube or bag containing anti-coagulant,
b) Isolating the PBMCs using FICOLL gradient centrifugation,
c) Seeding the peripheral blood mononuclear cells in the culture flask in the presence of RPMI 1640 cell culture media with 20% fetal bovine serum (FBS),
d) Incubating the cells in an incubator with 5% CO2 concentration for overnight at 37° C. temperature in an incubator,
e) Removing the non-adherent cells along with the culture media by gently shaking the flask after culturing overnight which is discarded or used for establishing secondary cultures by seeding in a new culture vessel,
f) Removing the adherent cells of the overnight PBMC cultures using accutase or 20 mM EDTA solution,
g) Collecting the content in a centrifuge tube and pellet the cells by centrifuging at 400 g for 15 minutes,
h) Washing the detached cells by resuspending them in Dulbecco's phosphate buffered saline, followed by optionally with RPMI 1640 medium and centrifuging at 400 g for 15 minutes,
i) Resuspending the cell pellet in MACS buffer and incubating the cells with anti-CD3 antibody conjugated magnetic beads (Miltenyi biotech) and carrying out positive selection of the CD3+ cells using the magnetic cell separation system wherein in this column-based separation, the CD3+ cells is retaining in the column and the CD3 negative cells is collected in the collection tube,
j) Collecting the CD3 negative cells and incubating it with anti-CD14 conjugated magnetic beads and carrying out the CD14 positive selection of CD3 negative population,
k) Collecting the CD3 negative-CD14 negative cells in the collection tube and obtaining the CD3 negative-CD14positive cells by eluting from the column, taking an aliquot of cells from each fraction and staining with flurochrome labelled antibodies such as CD3, CD14, CD56, CD19, CD4, CD8 and analysing using flow cytometer to check the cellular composition,
l) Culturing these CD3 negative-CD14 negative cells population in the culture vessel using the RPMI 1640 culture media with 20% fetal bovine serum for 3 days,
m) Removing the non-adherent cells at the end of 3 days of culturing, by washing adherent cells thrice using Dulbecco Phosphate Buffered Saline (DPBS) and adding the RPMI 1640 culture media containing 20% fetal bovine serum and interleukin-2 at a concentration of 20units/ml,
n) Harvesting the number of T cell generated in the nonadherent fraction in a centrifuge tube on day 6, centrifuging the cells to pellet it and then resuspending the cell pellet in fresh RPMI 1640 culture media containing 20% fetal bovine serum and interleukin-2 at a concentration of 20units/ml and seeding them in a separate culture vessel then incubating at 37° C. temperature in the presence of 5% CO2 for further expansion of the T cells to obtain large quantity of them, staining some cells for using flurochrome labelled antibodies CD3, CD14, CD56, CD19, CD4, CD8 and analysing using flow cytometer to understand the types of cells present in the purified population confirming the presence of CD3+ T cells in the culture, further adding fresh RPMI 1640 culture media containing 20% fetal bovine serum and interleukin-2 at a concentration of 20units/ml in to the parental flask containing adherent cells and continue culturing the adherent cells in the flask to generate more T cells,
o) Repeating the T cells collection step as described in step (n) once in every 2-3 days until the adherent cells disappear from CD3 negativeCD14 negative cell culture flask and only non-adherent T cells are observed in the culture,
p) Culturing T cells and expanding through cycles of proliferation by adding fresh growth medium RPMI 1640 culture media containing 20% fetal bovine serum or using serum free medium such as LymphoONE or X-VIVO medium and including interleukin-2 or IL-15 or IL-7 or any other T cell growth promoting cytokines or interleukins or growth factors once in every 2-3 days depending of the growth rate of cells,
q) Obtaining cells following steps i, j, k, and 1 and further fractionating through CD19+ cell depletion, CD56+ cell depletion and obtaining CD3-CD14-CD19-CD56-ve, CD3-CD14-CD19-CD56+ve, CD3-CD14-CD19+CD56-ve cells and use them for generating T cells,
r) Separating and purifying the cells through cell sorting using a cell sorter instrument as an alternative to magnetic bead-based separation of the desired cell population of interest such as mentioned in the step q wherein the cells obtained at k, 1, m, n, o, and p is used for therapeutic purposes with or without further manipulation.

Another aspect of the present invention provides a novel method of generation of chimeric antigen receptor (CAR) using the T cells generated from CD3-CD14-fraction comprising the steps of
a) Incubating the T cells obtained on or after day 6 with lentiviral particles containing CD19 CAR gene or any other CAR gene for 2 hours at 37° C. in 2 ml volume at desired ratio of cell:virus, mixing the cell suspension once in every 15 minutes intermittently and adding transduction enhancers such retronectin or popybrene to increase the transfection efficiency,
b) After 2 hours of incubation, adding 8 ml of culture media to the mixture and keeping the culture vessel back in the incubator and next day, pelleting the cells by collecting the culture vessel content in a centrifuge tube and centrifuging the tube at 400 g for 10 minutes,
c) Suspending the cell pellet in fresh RPMI medium with 20% FBS or serum free medium such as LymphoONE or X-VIVO medium and seeding in a culture vessel and incubating in an incubator with 37° C. and 5% CO2,
d) Adding IL-2 in the medium at the step c at a concentration of 50-100 IU/ml,
e) Collecting the cells from culture vessel every 2-3 days and pelleting the cells by centrifuging as mentioned in the step b and resuspending the cell pellet in fresh culture medium as described in step c and adding the IL-2 as mentioned in the step d, alternatively, after 2-3 days of culture, the ⅔rd of culture medium is removed when the cells are at the bottom and fresh culture medium is added and culture is continued,
f) Continuing the T cell culture by repeating the process mentioned in step e once in every 2-3 days, generating CAR T cells wherein alternatively, the T cells is grown in a closed system such as cell culture bags,
g) Injecting the cell into the same patient through intravenous route or into the tumor directly, wherein for the therapy it needs to be administered in an autologous manner, alternatively, mutating the TCR gene of T cells recovered from step at k, 1, m, n, o, and p using Crisper technology and making the T cell receptor of host cell non-functional through the mutation and then introducing the CAR gene of interest, wherein such cells lacking the endogenous T cell receptor can be used for treating patients in allogenic manner and the off-the-shelf CAR T cell therapeutic products can be developed.

### DESCRIPTION OF THE DRAWING

FIG. 1 illustrates the cells of different morphology observed in the culture of different fractions of cells obtained from overnight adherent peripheral blood mononuclear cells on day 1. A) CD3 negativeCD14 negative (CD3-CD14) fraction; B) CD3 negativeCD14positive (CD3-CD14+) fraction; 200× magnification.

FIG. 2 illustrates the characterization of the non-adherent cells obtained from CD3 negativeCD14 negative (CD3-CD14-) cell fraction on culture on day 6. It shows the presence of primarily CD3+ T cells and a small percentage of CD56+NK cells. All the CD3+ cells were also CD71+ which indicate their highly proliferative nature.

FIG. 3 illustrates the non-adherent cells obtained from the day 6 of CD3 negativeCD14 negative (CD3-CD14-) fraction culture showed presence of primarily CD3+CD4+ T cells and only a small fraction of CD4+ T cells expresses CD195+ on the surface.

FIG. 4 illustrates the proliferating T cells from the culture of CD3 negative CD14 negative fraction on 6.

FIG. 5 illustrates the measuring of the T-cell excision circles (Trec) from the DNA preparations of adherent/non adherent cells obtained from CD3-CD14+ cultures and CD3-CD14—cultures.

FIG. 6 illustrates the characterization of the stem cells in the overnight adherent fractions based on the expression of CD34, CD117 and CD14 markers. FIG. 6A illustrates the identification of the stem cells in the peripheral blood mononuclear cells of overnight adherent fraction. FIG. 6B illustrates the identification of the stem cells in the CD3-CD14—fraction. FIG. 6C illustrates the identification of the stem cells in the CD3-CD14+ fraction.

FIG. 7 illustrates the T cells obtained from the day 9 of CD3 negativeCD14 negative (CD3-CD14-) fraction culture transduced with lentiviral particles carrying CD19-specific CAR gene.

FIG. 8 illustrates the expression of CAR gene on the T cells on day 2 following transduction.

FIG. 9 illustrates the coculture of the CAR T cells with cancer. A) CAR T cells harboring CD19 specific CAR cocultured with K562 cells, B) CAR T cells harboring CD19 specific CAR cocultured with K562 cells expressing CD19 on the surface.

FIG. 10 illustrates the dot blot scatter and histogram showing two different CD19 specific CAR T cells killing and depleting the K562 cells expressing CD19 in the co-culture following staining with anti-CD19 antibody labelled with APC fluorochrome and analyzed using a flow cytometer.

FIG. 11 illustrates the dot blot scatter and histogram showing two different CD19 specific CAR T cells killing CD19 expressing K562 cells in the co-culture by staining the dead cells using propidium iodide staining and analyzing in a flow cytometer.

### DETAILED DESCRIPTION OF THE INVENTION

The present invention describes a novel method of generating the T cells from the cell fraction obtained from human peripheral blood progenitor cells or peripheral blood mononuclear cells (PBMC) or from its precursors in peripheral blood.

One of the embodiment of the present invention provides a novel method of generating T cells from PBMC or peripheral blood progenitor cells obtained from human blood.

Another embodiment of the present invention provides a novel method of generating T cells from the progenitors in the PBMC cultures and the CD3 negative CD14 negative fraction of overnight adherent cultures of PBMC.

Another embodiment of the present invention provides a novel method of generating chimeric antigen receptor, CAR T cells using the T cells from specific fraction of peripheral blood progenitor cells.

In order that the present disclosure can be more readily understood, certain terms are defined. As used in this application, except as otherwise expressly provided herein, each of the following terms shall have the meaning set forth below. Additional definitions are set forth throughout the application.

As used herein, the indefinite articles “a” or “an” should be understood to refer to “one or more” of any recited or enumerated component.

The terms “about” or “comprising essentially of refer to a value or composition that is within an acceptable error range for the particular value or composition as determined by one of ordinary skill in the art, which will depend in part on how the value or composition is measured or determined, i.e., the limitations of the measurement system. For example, “about” or “comprising essentially of can mean within 1 or more than 1 standard deviation per the practice in the art. Alternatively, “about” or “comprising essentially of can mean a range of up to 10% (i.e., ±10%). For example, about 3 mg can include any number between 2.7 mg and 3.3 mg (for 10%).

It is understood that wherever aspects are described herein with the language “comprising,” otherwise analogous aspects described in terms of “consisting of and/or “consisting essentially of are also provided.

“Administering” refers to the physical introduction of an agent to a subject, using any of the various methods and delivery systems known to those skilled in the art. Exemplary routes of administration for the T cells prepared by the methods disclosed herein include intravenous, intramuscular, subcutaneous, intraperitoneal, spinal or other parenteral routes of administration, for example by injection or infusion.

The phrase “parenteral administration” as used herein means modes of administration other than enteral and topical administration, usually by injection, and includes without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrasternal injection and infusion as well as in vivo electroporation. In some embodiments, the T cells prepared by the present methods is administered via a non-parenteral route, e.g., orally. Other non-parenteral routes include a topical, epidermal or mucosal route of administration, for example, intranasally, vaginally, rectally, sublingually or topically. Administering can also be performed, for example, once, a plurality of times, and/or over one or more extended periods.

“T-cells” refers to several types of T-cells, namely: Helper T-cells {e.g., CD4+ cells, effector T cells, T helper-1 or Th1, T helper-2 or Th2 cells, regulatory T cells), Cytotoxic T-cells (also known as TC, cytotoxic T lymphocyte, CTL, T-killer cell, cytolytic T cell, CD8+ T-cells or killer T cell), Memory T-cells ((i) stem memory TSCM cells, like naive cells, are CD45RO−, CCR7+, CD45RA+, CD62L+(L-selectin), CD27+, CD28+ and IL-7Ra+, but they also express large amounts of CD95, IL-2RP, CXCR3, and LFA-1, and show numerous functional attributes distinctive of memory cells); (ii) central memory TCM cells express L-selectin and are CCR7 and CD45RO+ and they secrete IL-2, but not IFNy or IL-4, and (iii) effector memory TEM cells, however, do not express L-selectin or CCR7 but do express CD45RO and produce effector cytokines like IFNy and IL-4), Regulatory T-cells (Tregs, suppressor T cells, or CD4+CD25+ regulatory T cells), Natural Killer T-cells (NKT), and Gamma Delta T-cells. T cells found within tumors are referred to as “tumor infiltrating lymphocytes” or “TIL.” B-cells, on the other hand, play a principal role in humoral immunity (with antibody involvement). It makes antibodies and antigens and performs the role of antigen-presenting cells (APCs) and turns into memory B-cells after activation by antigen interaction. In mammals, immature B-cells are formed in the bone marrow, where its name is derived from.

Cell “proliferation,” as used herein, refers to the ability of T cells to grow in numbers through cell division. Proliferation can be measured by staining cells with carboxyfluorescein succinimidyl ester (CFSE). Cell proliferation can occur in vitro, e.g., during T cell culture, or in vivo, e.g., following administration of a T cell therapy.

The term “genetically engineered,” “gene editing,” or “engineered” refers to a method of modifying the genome of a cell, including, but not limited to, deleting a coding or non-coding region or a portion thereof or inserting a coding region or a portion thereof. In some embodiments, the cell that is modified is a lymphocyte, e.g., a T cell, which can either be obtained from a patient or a donor. The cell can be modified to express an exogenous construct, such as, e.g., a chimeric antigen receptor (CAR) or a T cell receptor (TCR), which is incorporated into the cell's genome.

A “therapeutically effective amount” or “therapeutically effective dosage,” as used herein, refers to an amount of the T cells or the DC cells that are produced by the present methods and that, when used alone or in combination with another therapeutic agent, protects a subject against the onset of a disease or promotes disease regression evidenced by a decrease in severity of disease symptoms, an increase in frequency and duration of disease symptom-free periods or a prevention of impairment or disability due to the disease affliction. The ability of the T cells or DC cells to promote disease regression can be evaluated using a variety of methods known to the skilled practitioner, such as in human subjects during clinical trials, 10 in animal model systems predictive of efficacy in humans or by assaying the activity of the agent in in-vitro assays.

“Treatment” or “treating” of a subject refers to any type of intervention or process performed on, or the administration of one or more T cells prepared by the present invention to the subject with the objective of reversing, alleviating, ameliorating, inhibiting, slowing down or preventing the onset, progression, development, severity or recurrence of a symptom, complication or condition, or biochemical indicia associated with a disease. In one embodiment, “treatment” or “treating” includes a partial remission. In another embodiment, “treatment” or “treating” includes a complete remission.

As described herein, any concentration range, percentage range, ratio range or integer range is to be understood to include the value of any integer within the recited range and when appropriate, fractions thereof (such as one-tenth and one-hundredth of an integer), unless otherwise indicated.

One of the most preferred embodiment of the present invention is to provide a novel method of generating T cells from the peripheral blood progenitor cells comprising the steps of
a) Collecting blood from donor in blood collection tube or bag containing anti-coagulant,
b) Isolating the PBMCs using FICOLL gradient centrifugation or any other method or any methods using the specialized equipment and reagents,
c) Seeding the peripheral blood mononuclear cells in the culture flask in the presence of RPMI 1640 cell culture media with 20% fetal bovine serum (FBS) with or without 10% human serum or plasma; if serum free media is used, fetal bovine serum or plasma can be avoided,
d) Incubating the cells in an incubator with 5% CO2 concentration for overnight at 37° C. temperature in an incubator.
e) Removing the non-adherent cells along with the culture media by gently shaking the flask after culturing overnight which can be discarded or used for establishing secondary cultures by seeding in a new culture vessel, f) Removing the adherent cells of the overnight PBMC cultures using acutase or EDTA solution or any other cell detachment or cell dissociation reagents or methods,
g) Collecting the content in 50 ml tube and pellet the cells by centrifuging at 400 g for 15 minutes,
h) Washing the detached cells by resuspending them in Dulbecco's phosphate buffered saline or RPMI 1640 medium with or without fetal bovine serum and centrifuging at 400 g for 15 minutes,
i) Resuspending the cell pellet in MACS buffer and incubate the cells with anti-CD3 antibody conjugated magnetic beads and carrying out positive selection of the CD3+ cells using the magnetic cell separation system (Miltenyi biotech) wherein in this column-based separation, the CD3+ cells is retaining in the column and the CD3 negative cells is collected in the collection tube,
j) Collecting the CD3 negative cells and incubating it with anti-CD14 conjugated magnetic beads and carrying out the CD14 positive selection of CD3 negative population,
k) Collecting the CD3 negative-CD14 negative cells in the collection tube and obtaining the CD3 negative-CD14positive cells by eluting from the column, Staining some cells with flurochrome labelled antibodies such as CD3, CD14, CD56, CD19, CD4, CD8, etc and analysing using flow cytometer to understand the types of cells present in the purified population,
l) Culturing these CD3 negative-CD14 negative cells population of cells in the culture vessel using the RPMI 1640 culture media with 20% fetal bovine serum for 3 days,
m) At the end of 3 days of culturing, removing the non-adherent cells by gently washing thrice using Dulbecco Phosphate Buffered Saline (DPBS) and adding the RPMI 1640 culture media containing 20% fetal bovine serum and interleukin-2 at a concentration of 20units/ml wherein alternatively, other cytokines such as IL-15 or IL-7 or any other T cell growth promoting cytokines or interleukins or growth factors, and other cell culture media including serum free can be used,
n) On day 6, harvesting the large number of T cell generated, centrifuging the cells to pellet it and then resuspending the cell pellet in fresh RPMI 1640 culture media containing 20% fetal bovine serum and interleukin-2 at a concentration of 20units/ml and seeding them in a separate culture vessel then incubating at 37° C. temperature in the presence of 5% CO2 for further expansion of the T cells to obtain large quantity of them wherein alternatively, other interleukins such as IL-15, IL-7 or any other T cell growth promoting cytokines or interleukins or growth factors can be used, staining some cells with flurochrome labelled antibodies CD3, CD14, CD56, CD19, CD4, CD8, etc and analysing using flow cytometer to understand the types of cells present in the purified population confirming the presence of CD3+ T cells in the culture, further adding fresh RPMI 1640 culture media containing 20% fetal bovine serum and interleukin-2 at a concentration of 20units/ml in to the flask and continuing culturing the adherent cells in the flask to generate more T cells,
o) Repeating the T cells collection step as described in step (n) once in every 2-3 days until the adherent cells disappear from CD3 negativeCD14 negative cell culture flask and only non-adherent T cells are observed in the culture,
p) Culturing T cells are and expanding through cycles of proliferation by adding fresh growth medium RPMI 1640 culture media containing 20% fetal bovine serum or using serum free medium such as LymphoONE (Takara Bio) or X-VIVO medium (Lonza) and including interleukin-2 or IL-15 or IL-7 or any other T cell growth promoting cytokines or interleukins or growth factors once in every 2-3 days depending of the growth rate of cells,
q) Obtaining cells following steps i, j, k, and 1 and further fractionating through CD19+ cell depletion, CD56+ cell depletion and obtaining CD3-CD14-CD19-CD56-ve, CD3-CD14-CD19-CD56+ve, CD3-CD14-CD19+CD56-ve cells and use them for generating T cells,
r) Separating and purifying the cells through cell sorting using a cell sorter instrument as an alternative to magnetic bead-based separation of the desired cell population of interest such as mentioned in the step q wherein the cells obtained at k, 1, m, n, o, and p is used for therapeutic purposes with or without further manipulation.

Another embodiment of the present invention relates to the method of generating T cells from the peripheral blood progenitor cells wherein, culture media is supplemented with 10% human serum, human plasma, fetal bovine serum.

Another embodiment of the present invention relates to the method of generating T cells from the peripheral blood progenitor cells wherein, the methods used for isolating PBMCs as per step b can be selected from isolation by FICOLL gradient centrifugation, isolation by cell preparation tubes and isolation by SepMate tubes or any other instrument to isolate the PBMCs known in the art.

Another embodiment of the present invention relates to the method of generating T cells from the peripheral blood progenitor cells wherein, the culture media is selected from AIM V media, RPMI 1640, DMEM—Dulbecco's Modified Eagle Medium, Dulbecco's Modified Eagle Medium: Nutrient Mixture F-12, F10 Nutrient Mixture, Ham's F12 Nutrient Mixture, Media 199, Minimum Essential Media, RPMI Medium 1640, Opti-MEM I Reduced Serum Media, LymphoOne medium, X-VIVO medium, Iscove's Modified Dulbecco's Medium, mammalian cell culture medium.

Another embodiment of the present invention relates to the method of generating T cells from the peripheral blood progenitor cells wherein, the concentration of fetal bovine serum is selected from the range of 5 to 40%.

Another embodiment of the present invention relates to the method of generating T cells from the peripheral blood progenitor cells wherein in step m and n, cytokines such as IL-15, IL-7, T cell growth promoting cytokines, interleukins and growth factors is used.

Another preferred embodiment of the present invention provides a novel method of generating chimeric antigen receptor (CAR) using the T cells generated from specific fraction comprising the steps of
a) Incubating the T cells obtained on or after day 6 with lentiviral particles containing CD19 CAR gene or any other CAR gene for 2 hours at 37° C. in 2 ml volume at desired ratio of cell:virus, mixing the cell suspension once in every 15 minutes intermittently and adding of transduction enhancers such retronectin or popybrene are optional step to increase the transfection efficiency wherein alternative to lentiviral vector, other vectors such as plasmid, adeno-associated virus, transposons, etc is used for transferring the CAR gene into the T cells and making the CAR T cells,
b) After 2 hours of incubation, adding 8 ml of culture media to the mixture and keeping the culture vessel back in the incubator and next day, pelleting the cells by collecting the culture vessel content in a centrifuge tube and centrifuging the tube at 400 g for 10 minutes,
c) Suspending the cell pellet in fresh RPMI medium with 20% FBS or serum free medium such as LymphoONE (Takara Bio) or X-VIVO medium (Lonza) and seeding in a culture vessel and incubating in an incubator with 37° C. and 5% CO2,
d) Adding IL-2 in the medium at the step c at a concentration of 50-100 IU/ml, alternatively, IL-15, IL-7 or other T cell growth promoting cytokines
e) Collecting the cells from culture vessel every 2-3 days and pelleting the cells by centrifuging as mentioned in the step b and resuspending the cell pellet in fresh culture medium as described in step c and adding the IL-2 as mentioned in the step d, alternatively, after 2-3 days of culture, the ⅔rd of culture medium is removed when the cells are at the bottom and fresh culture medium is added and culture is continued,
f) Continuing the T cell culture by repeating the process mentioned in step e once in every 2-3 days, generating enough number of CAR T cells for therapeutic purposes, alternatively, the T cells is grown in a closed system such as cell culture bags.
g) Injecting the cell into the same patient through intravenous route or into the tumor directly, wherein for the therapy it needs to be administered in an autologous manner, alternatively, mutating the TCR gene of T cells recovered from step at k, 1, m, n, o, and p using Crisper technology and making the T cell receptor of host cell non-functional through the mutation and then introducing the CAR gene of interest, wherein such cells lacking the endogenous T cell receptor can be used for treating patients in allogenic manner and the off-the-shelf CAR T cell therapeutic products can be developed.

As per another embodiment the present invention provides analysing by staining with flurochorme labelled antibodies that can bind to the CAR molecule and analysing in the flow cytometer and determining percentage of T cells expression, alternatively, using molecular biological assays such as quantitative PCR of digital PCR for measuring the CAR gene harbouring T cells,

Another embodiment of the present invention relates to the method of generating CAR T cells wherein vector for transferring the CAR gene into the T cells is selected from lentiviral particles, plasmid, adeno-associated virus and transposons.

As per another embodiment of the present invention, the concentration of interleukin is selected from the range of 5 to 6000 IU/ml.

The following examples are illustrated to describe the scope of the invention.

### EXAMPLES

### Example 1: Observations During Generation of Lymphoid and Myeloid Cell Generation Niche, and the T Cell from the Fraction of Peripheral Blood Mononuclear Cells

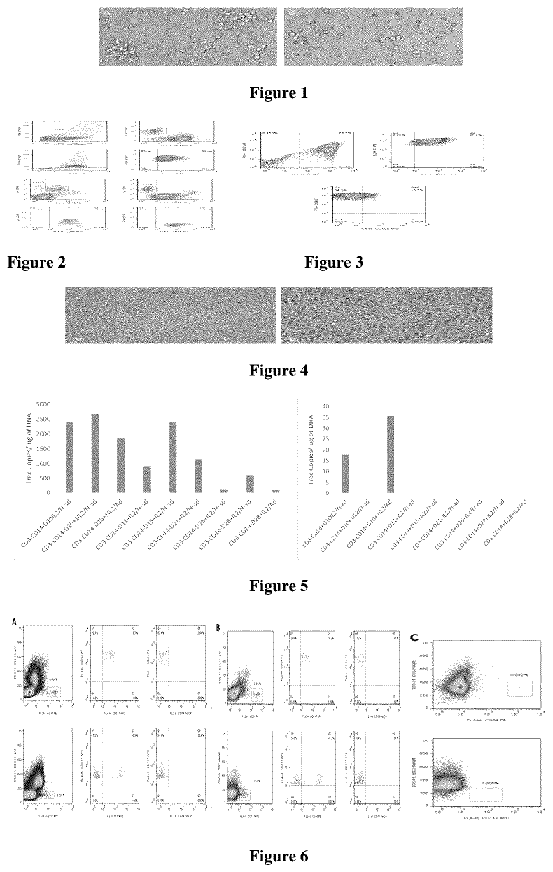

Culturing cells: PBMC was isolated from the blood using ficoll gradient centrifugation and seeded them in the culture flasks and cultured them overnight. Briefly, the blood was diluted and overlaid on the ficoll solution. PBMC was cultured using RPMI-1640 medium supplemented with 20% fetal bovine serum and 10% human serum or plasma (growth medium). Human plasma or serum can be optional for short term cultures and T cell generation can be seen even in the absence of human plasma or serum. The flasks seeded with peripheral blood mononuclear cells 30 million cells/T25 cm2 flask/8 ml growth medium. The culture vessel was incubated in an incubator maintaining 37° C. and 5% CO2. The obtained overnight adherent cell fraction was analyzed in the flow cytometer. To identify the cells with T cell generation potential from this mixed population, firstly depleted all the T cells using the anti-CD3 magnetic beads and Miltenyi magnetic bead separation system. The CD3 negative population were taken and subjected to CD14 positive selection and then obtained the CD3 negativeCD14positive cells and the CD3 negativeCD14 negative cells. These two populations of cells were cultured separately seeding at a density of 2 million cells in each 25 cm 2 culture vessel with RPMI medium containing 20% FBS and followed up in the culture. On day 3, all the non-adherent cells were removed from the culture and then washed them thrice with Dulbecco phosphate buffered saline (DPBS) and added the fresh RPMI with 20% FBS and 20 units of IL2 per ml of medium. The flasks were observed regularly everyday under inverted microscope and images were captured using the camera attached to the microscope. Use of the same medium supplemented with fetal bovine or any serum/plasma, and human serum/plasma; or serum free medium with or without any supplementation to de novo generate T cells from its peripheral blood mononuclear cells using the described method or a modified version of the method described in this invention either in the presence or absence of any other cytokines or biological or chemical molecules. Results were represented in FIG. 1A, 1B, 2, 3, 4A, 4B. 7A, 7B, 9A, and 9B.

Bead separation of cells: For separation of different fraction of cells, Miltenyi magnetic beads were used following the manufacturers protocol. From the overnight adherent cells, CD3+ cells were selected positively and the remaining CD3 negative cells were also collected. The CD3 negative cells were subjected to CD14 positive selection. Finally, the CD3-CD14+ cells and CD3-CD14-cell population were obtained. These two fractions of cells were cultured to understand the T cell producing potential. Results were represented in FIGS. 1A, 1B, 6A, 6B, and 6C.

Analysis of cell by flow cytometer: Harvested cells were centrifuged, suspended in RPMI-20%, counted, and 50 μl aliquots were incubated with fluorochrome-labelled antibodies for 20 minutes at room temperature in the dark. One ml of DPBS containing 1% FBS and 0.09% sodium azide (wash buffer) was added, tubes were briefly vortexed, and then 250 μl of fixative (9.25% formaldehyde plus 3.75% methanol) was added, followed by 3 ml of wash buffer. Following centrifugation, cells were washed an additional time, then suspended in 1% paraformaldehyde and acquired on a FACS Calibur cytometer or Cytoflex cytometer (Beckman Coulter). Ten-to-hundred thousand events were acquired per tube, depending on the frequency of populations of interest. Analyses were carried out using the CellQuest and Cytoflex software programs. Results were represented in FIGS. 1H, 2A, 3A, 4C, 4D and 5F. Flow cytometry-based analysis of the fraction revealed that the overnight adherent cells had cells with stem cell markers, particularly the CD3-CD14-fraction was found to be enriched for stem cells as given below in table.

TABLE 1
Presence of stem cells based on the expression of CD34 and CD117
markers in different fractions of overnight adherent cells,
CD3-CD14- fraction of PCMC, and CD3-CD14+ fraction.
CD34+ stem cells

| Cells | Bc307 (34 + 117 + | Bc130 (34 + 117 + |
|---|---|---|
| | [%]/34 + 117 − [%]) | [%]/34 + 117 − [%]) |
| Pbmc | 0.086 (80/20) | 0.173 (30/70) |
| (ON-Ad) | | |
| CD3-14− | 0.513 (70/30) | 0.554 (55/45) |
| CD3-14+ | 0 | 0 |

CD117+ stem cells

| | | |
|---|---|---|
| Cells | Bc307 (117 + 34 + | Bc130 (117 + 34 + |
| | [%]/117 + 34 − [%]) | [%]/117 + 34 − [%]) |
| Pbmc | 0.201 (30/70) | 0.128 (34/66) |
| (ON-Ad) | | |
| CD3-14− | 1.11 (40/60) | 0.818 (38/62) |
| CD3-14+ | 0 | 0 |

The non-adherent cells were harvested from the cultured CD14 negative cell flask, stained with different antibody markers and analyzed in a flow cytometer. It was found that about 68% of the cells were CD3+ cells, 9% were NK cells, and remaining were other cells. Majority of the NK cells were CD56dimCD16+CD71-cells. Interestingly, it was found that all the CD3+ T cells were CD71+, which is the transferrin receptor, a marker for proliferating cells. Thus, these T cells appear to be highly proliferative in nature. Results were represented in FIG. 2. Further characterization of these CD3+ T cells revealed that they are primarily CD4+ cells (>76%) and they are all CD25+ and only a small fraction is CD195+. Results were represented in FIG. 3. The study indicates that the overnight adherent population, particularly the CD3-CD14-fraction contains the precursors of T cells. These precursor cells within this CD3-14-fraction ideal for transplantation or generating normal T cells for therapeutic purposes or generating genetically modified T cells such as Chimeric antigen receptor (CAR) T cells or gene edited T cells for therapeutic purposes.

Quantitation of TREC using quantitative PCR: A molecular construct was developed to provide for TREC copy number quantitation. A 408 bp fragment of DNA containing the dRec-yJa signal-joint break point was amplified from human uncultured PBMC DNA and inserted into the pCR2.1-TOPO vector (Invitrogen). The primers used for this were: 5-AAAGAGGGCAGCCCTCTCCAAGGCAAAA-3 (sense) and 5-ACTTCCATCGCAATTCAGGACTCACTT-3 (antisense). DNA was purified from cultured cells using the DNeasy Blood & Tissue Kit. The PCR reactions were performed in a total volume of 25 ul that contained 12.5 ul of SYBR Green/ROX master mix, 10.5 ml of water, 0.5 ml of each primer, and 1 ul of DNA. The primers used in these reactions were: 5′-CGTGAGAACGGTGAATGAAGAGCAGACA-3′ (sense) and 5′-CATCCCTTTCAACCATGCTGACACCTCT-3′ (antisense).

Samples from the same experiment were run in duplicate on the same plate, along with a dilution series of TREC plasmid DNA, and positive and negative control samples. DNA from TREC+ve PBMC and DNA from the Hela cell line were used as positive and negative controls, respectively. Genomic and plasmid DNA were handled in separate rooms and care was taken to ensure no contamination of reagents and assay materials with plasmid DNA. The reactions and analyses were carried out using an ABI Prism 7500 real-time PCR instrument. Values were extrapolated to TREC copy number per mg DNA. Results were represented in FIGS. 5A and 5B.

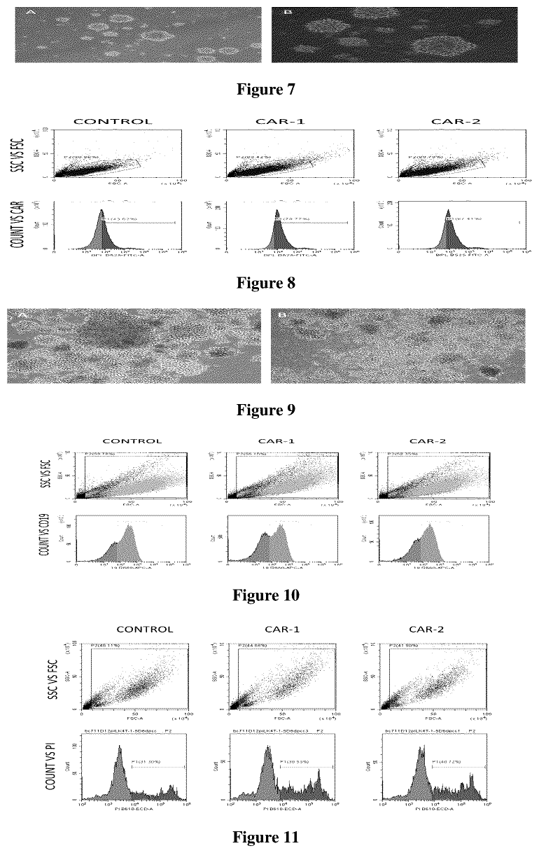

Example 2: Generation of Chimeric Antigen Receptor (CAR) T cells: The T cells obtained on or after day 6 were mixed with lentiviral particles containing CD19 CAR gene for 2 hours in a culture vessel at 37° C. in an incubator. The mixture was mixed gently once in every 15 minutes. The transduction enhancers such retronectin or popybrene were added to increase the transfection efficiency. After 2 hours of incubation, 8 ml of culture media was added to the mixture and the culture vessel was kept back in the incubator. Next day, the cells were pelleted by collecting the culture vessel content in a centrifuge tube and centrifuging the tube at 400 g for 10 minutes. Results were represented in FIGS. 7A and 7B. During the culture, an aliquot of cells was taken and stained with anti-Fab-FITC antibody and analysed in a flow cytometer. The cells were also stained with specific individual and combination of antibodies to detect the CD3, CD4 and CAR expression by flow cytometer tubes. The expression of CAR gene on the T cells was confirmed by flow cytometry. Results were represented in FIG. 8.

Culturing and expansion of the T cells: The cell pellet was suspended in fresh RPMI medium with 20% FBS and incubated in an incubator with 37° C. and 5% CO2. Alternatively, the cells can be suspended in serum free medium such as LymphoONE (Takara Bio) or X-VIVO medium (Lonza) and for culturing the T cells. IL-2 was added in the medium at a concentration of 50-100 IU/ml. Alternatively, IL-15, IL-7 or other T cell growth promoting cytokines can also be used. The T cells from culture vessel were collected every 2-3 days and the cells were pelleted by centrifugation and resuspended in fresh culture medium and culture was continued. The T cell culture was continued by repeating this process once in every 2-3 days to generate large number of CAR T cells for therapeutic purposes. Results were represented in FIGS. 9A and 9B.

Functional analysis of CAR T cells: Functional activity of CAR T cells was studied by measuring its ability to kill the K562 cells expression CD19 on the surface. The K562-19 cells were cocultured with the CAR T cells. The cocultured cells were stained for flurochrome labelled CD19 antibody to measure the depletion of K562-19 cells. The cocultured cells were also stained using propidium iodide to measure the dead cells in the culture. The stained cells were acquired in a flow cytometer and the data was analyzed and represented in the form of dot blot or histogram. The study showed that the CAR T cells generated in CD3-CD14-fraction have the functional ability to kill the cancer cells in a specific manner They can specifically recognize the CD19 molecule on the cancer cells and kill them. In the coculture, a specific decline in the CD19+ve cells and an increase in the dead cell population were observed clearly. Results were represented in FIGS. 10 and 11.

1. A novel method of generating T cells from the peripheral blood progenitor cells comprising the steps of
a) Collecting blood from donor in blood collection tube or bag containing anti-coagulant,
b) Isolating the PBMCs using FICOLL gradient centrifugation,
c) Seeding the peripheral blood mononuclear cells in the culture flask in the presence of RPMI 1640 cell culture media with 20% fetal bovine serum (FBS),
d) Incubating the cells in an incubator with 5% CO2 concentration for overnight at 37° C. temperature in an incubator,
e) Removing the non-adherent cells along with the culture media by shaking the flask after culturing overnight which is discarded or used for establishing secondary cultures by seeding in a new culture vessel,
f) Removing the adherent cells of the overnight PBMC cultures using accutase or 20 mM EDTA solution,
g) Collecting the content in a centrifuge tube and pellet the cells by centrifuging at 400 g for 15 minutes,
h) Washing the detached cells by resuspending them in Dulbecco's phosphate buffered saline, followed by optionally with RPMI 1640 medium and centrifuging at 400 g for 15 minutes,
i) Resuspending the cell pellet in MACS buffer and incubating the cells with anti-CD3 antibody conjugated magnetic beads and carrying out positive selection of the CD3+ cells using the magnetic cell separation system wherein in this column-based separation, the CD3+ cells is retaining in the column and the CD3 negative cells is collected in the collection tube,
j) Collecting the CD3 negative cells and incubating it with anti-CD14 conjugated magnetic beads and carrying out the CD14 positive selection of CD3 negative population,
k) Collecting the CD3 negative-CD14 negative cells in the collection tube and obtaining the CD3 negative-CD14positive cells by eluting from the column, taking an aliquot of cells from each fraction and staining with flurochrome labelled antibodies such as CD3, CD14, CD56, CD19, CD4, CD8 and analysing using flow cytometer to check the cellular composition,
l) Culturing these CD3 negative-CD14 negative cells population of cells in the culture vessel using the RPMI 1640 culture media with 20% fetal bovine serum for 3 days,
m) At the end of 3 days of culturing, removing the non-adherent cells by washing adherent cells thrice using Dulbecco Phosphate Buffered Saline (DPBS) and adding the RPMI 1640 culture media containing 20% fetal bovine serum and interleukin-2 at a concentration of 20units/ml,
n) On day 6, harvesting the number of T cell generated in the nonadherent fraction in a centrifuge tube, centrifuging the cells to pellet it and then resuspending the cell pellet in fresh RPMI 1640 culture media containing 20% fetal bovine serum and interleukin-2 at a concentration of 20units/ml and seeding them in a separate culture vessel then incubating at 37° C. temperature in the presence of 5% CO2 for further expansion of the T cells, staining some cells with flurochrome labelled antibodies CD3, CD14, CD56, CD19, CD4, CD8 and analysing using flow cytometer confirming the presence of CD3+ T cells in the culture, adding fresh RPMI 1640 culture media containing 20% fetal bovine serum and interleukin-2 at a concentration of 20units/ml in to the parental flask containing adherent cells and continue culturing the adherent cells in the flask to generate T cells,
o) Repeating the T cells collection step as described in step (n) once in every 2-3 days until the adherent cells disappear from CD3 negativeCD14 negative cell culture flask and only non-adherent T cells are observed in the culture,
p) Culturing T cells and expanding through cycles of proliferation by adding fresh growth medium RPMI 1640 culture media containing 20% fetal bovine serum or using serum free medium such as LymphoONE or X-VIVO medium and including interleukin-2 or IL-15 or IL-7 or any other T cell growth promoting cytokines or interleukins or growth factors once in every 2-3 days depending of the growth rate of cells,
q) Obtaining cells following steps i, j, k, and 1 and further fractionating through CD19+ cell depletion, CD56+ cell depletion and obtaining CD3-CD14-CD19-CD56-ve, CD3-CD14-CD19-CD56+ve, CD3-CD14-CD19+CD56-ve cells for generating T cells,
r) Separating and purifying the cells through cell sorting using a cell sorter instrument as an alternative to magnetic bead-based separation of the desired cell population of interest such as mentioned in the step q.

2. The method as claimed in claim 1, wherein culture media in step c is supplemented with 10% human serum, human plasma, fetal bovine serum.

3. The method as claimed in claim 1, wherein the methods used for isolating PBMCs as per step b can be selected from isolation by FICOLL gradient centrifugation, isolation by cell preparation tubes and isolation by SepMate tubes.

4. The method as claimed in claim 1, wherein the culture media is selected from AIM V media, RPMI 1640, DMEM—Dulbecco's Modified Eagle Medium, Dulbecco's Modified Eagle Medium: Nutrient Mixture F-12, F10 Nutrient Mixture, Ham's F12 Nutrient Mixture, Media 199, Minimum Essential Media, RPMI Medium 1640, Opti-MEM I Reduced Serum Media, LymphoOne medium, X-VIVO medium, Iscove's Modified Dulbecco's Medium, mammalian cell culture medium.

5. A novel method of generating of chimeric antigen receptor (CAR) comprising the steps of
a) Incubating the T cells obtained with vector containing CD19 CAR gene or any other CAR gene for 2 hours at 37° C. in 2 ml volume at desired ratio of cell:virus, mixing the cell suspension once in every 15 minutes intermittently, adding of transduction enhancers such retronectin or popybrene to increase the transfection efficiency,
b) After 2 hours of incubation, adding 8 ml of culture media to the mixture and keeping the culture vessel back in the incubator and next day, pelleting the cells by collecting the culture vessel content in a centrifuge tube and centrifuging the tube at 400 g for 10 minutes,
c) Suspending the cell pellet in fresh RPMI medium with 20% FBS or serum free medium such as LymphoONE or X-VIVO medium and seeding in a culture vessel and incubating in an incubator with 37° C. and 5% CO2,
d) Adding IL-2 in the medium at the step c at a concentration of 50-100 IU/ml, e) Collecting the cells from culture vessel every 2-3 days and pelleting the cells by centrifuging as mentioned in the step b and resuspending the cell pellet in fresh culture medium as described in step c and adding the IL-2 as mentioned in the step d, optionally after 2-3 days of culture, the ⅔rd of culture medium is removed when the cells are at the bottom and fresh culture medium is added and culture is continued,
f) Continuing the T cell culture by repeating the process mentioned in step e once in every 2-3 days, generating CAR T cells wherein optionally the T cells is grown in a closed system such as cell culture bags.

6. The method as claimed in claim 5, wherein vector for transferring the CAR gene into the T cells is selected from lentiviral particles, plasmid, adeno-associated virus and transposons.

7. The method as claimed in claim 5, wherein the concentration of fetal bovine serum is selected from the range of 5 to 40%.

8. The method as claimed in claim 5, wherein in step m and n, cytokines such as IL-15, IL-7, T cell growth promoting cytokines, interleukins and growth factors is used.

9. The method as claimed in claim 5, wherein the concentration of interleukin is selected from the range of 5 to 6000 IU/ml.
